(19) United States
(12) Patent Application Publication
(10) Pub. No.: US 2023/0173075 A1
(43) Pub. Date: Jun. 8, 2023

(54) ENCAPSULATED COMPOSITIONS AND USE OF OCTENYLSUCCINIC ANHYDRIDE STARCH AS EMULSIFYING AGENT

(71) Applicant: Kansas State University Research Foundation, Manhattan, KS (US)

(72) Inventors: Yong-Cheng Shi, Manhattan, KS (US); Zhenhua Sun, Manhattan, KS (US)

(21) Appl. No.: 17/997,276

(22) PCT Filed: Apr. 29, 2021
(86) PCT No.: PCT/US2021/029823

§ 371 Date: Oct. 27, 2022

Related U.S. Application Data
(60) Provisional application No. 63/017,916, filed on Apr. 30, 2020.

(51) Int. Cl. A61K 47/36; A61K 31/216; A61K 31/12; A61K 9/16; A61K 9/107; A61K 47/44; B01J 13/04

(52) U.S. Cl. CPC A61K 47/36; A61K 31/216; A61K 31/12; A61K 9/1617; A61K 9/1652; A61K 9/1075; A61K 47/44; B01J 13/043

(57) ABSTRACT

Compounds encapsulated with octenylsuccinic anhydride modified starch and methods of forming the encapsulated compounds are provided. Poorly water-soluble compounds, especially pharmaceuticals, are solubilized within an oil phase, which is then formed into an emulsion with an aqueous phase comprising the OSA modified starch. The resulting emulsion can be dried, such as through spray drying, for form a powder comprising the encapsulated compound.

### RELATED APPLICATION

This application claims the priority benefit of U.S. Provisional Patent Application No. 63/017,916, filed Apr. 30, 2020, which is incorporated by reference herein in its entirety.

### BACKGROUND OF THE INVENTION

### Field of the Invention

Generally, the present invention is directed toward compositions encapsulated with octenylsuccinic anhydride (OSA) modified starch and methods of forming the encapsulated compounds. It has been discovered that poorly water-soluble compounds, especially pharmaceutical compounds, can be solubilized within an oil phase, which is then formed into an emulsion with an aqueous phase comprising the OSA modified starch. The resulting emulsion can be dried, such as through spray drying, and a powder comprising the encapsulated compound recovered.

### Description of the Prior Art

The Biopharmaceutics Classification System (BCS) is a scientific framework, first introduced in 1995, categorizing drug substances according to their water solubility and intestinal membrane permeability. According to the BCS, drug substances are divided into high/low solubility and permeability classes as follow: Class I (high solubility and high permeability), Class II (low solubility and high permeability), Class III (high solubility and low permeability), and Class IV (low solubility and low permeability).

There is great progress in drug discovery and research, but many new drugs fail to be commercially successful due to unacceptable safety (risk-to-benefit ratio), lack of efficacy, formulation, and market. Major reasons for the failure of new drug candidates are poor water solubility and modest absorption, which relates to many issues such as low oval bioavailability and increased cost of the drug product. Therefore, it is crucially important to enhance the solubility performance of poorly water-soluble drugs in formulation development.

Fenofibrate (FF), a poorly water soluble (<0.5 mg/L) and highly lipophilic drug, belongs to BCS class II drugs with good permeability but low oral bioavailability. FF in a hard gelatin capsule was originally launched in 1975 with the maximum bioavailability 60%. In the global market, FF (marketed as Tricor®, Lipofen® and Fenoglide®) has been widely used to reduce the levels of low-density lipoprotein-cholesterol and triglyceride, and raise high-density lipoprotein-cholesterol levels in blood, lowering the risk of cardiovascular disease, type 2 diabetes, and renal disease. Pharmaceutical drug performance could be controlled and better designed depending on its molecular structure, and physical state (crystalline or amorphous), where amorphous drugs exhibit a higher dissolution rate than the crystalline substances but worse stability. One type of formulation developed for FF (Lipofen®) is a hard gelatin capsule containing a mixture of a lipid and FF with hydroxypropyl methylcellulose. However, lipid formulations may leach into and interact with the capsule shells, causing brittleness or softness of the capsule shell, leakage of the filling and precipitation of the drug. In oral drug delivery, the drug and the drug carrier have to pass through the stomach with its low pH, which tends to affect the drug stability and the drug solubility as well as the properties of the drug carrier system.

A solid form of self-microemulsifying drug delivery system (solid SEMDDS, a lipid-based drug delivery) has been developed with solid carrier to improve the oral bioavailability of FF. Solid SMEDDS is an anhydrous system consisting of natural or synthetic oil(s), surfactant(s) and cosurfactant(s) or cosolvent(s) incorporated with the lipophilic drug in suitable proportions. However, there exist some limitations associated with SMEDDS. Only small molecule surfactants can be used to prepare SMEDDS. As the formulations of the emulsion always include a great amount of surfactant and co-surfactant, which may cause hemolysis or histopathological alterations of the tissue, disrupt normal membrane structure and may thus lead to cytotoxicity.

Therefore, a need exists in the art for a delivery system for poorly water-soluble compounds, especially pharmaceutical compounds, that preserve the stability and improve the bioavailability of the compound.

### SUMMARY OF THE INVENTION

According to one embodiment of the present invention there is provided an encapsulated composition comprising a poorly water-soluble compound, such as an active pharmaceutical ingredient (API), an oil, and an octenylsuccinic anhydride (OSA) modified starch. In a further embodiment, the weight ratio of starch to oil within the composition is 1:1 or less, and/or the poorly water-soluble compound comprises at least 1% by weight of the encapsulated composition.

According to another embodiment of the present invention there is provided an emulsion comprising a poorly water-soluble compound that is dispersed in an oil phase, and an aqueous phase comprising an octenylsuccinc anhydride (OSA) modified starch. In a further embodiment, the weight ratio of starch to oil within the emulsion is 1:1 or less.

According to still another embodiment of the present invention there is provided a method of forming an encapsulated composition. An oil phase comprising a poorly water-soluble compound solubilized in an oil is formed, as is an aqueous phase comprising an octenylsuccinic anhydride (OSA) modified starch dissolved in water. A quantity of the oil phase and a quantity of the aqueous phase are then mixed to form an emulsion. The weight ratio of starch to oil within the composition is 1:1 or less. The emulsion is then dried to form a powder comprising particles of the poorly water-soluble compound encapsulated in the OSA modified starch. In a further embodiment, the poorly water-soluble compound comprises at least 3% by weight of the powder.

### BRIEF DESCRIPTION OF THE DRAWINGS

FIGS. 1A and 1B are graphs of the density of spray-dried encapsulates made with (A) MCT and OS starch and (B) Capryol 90 and OS starch, respectively; and

FIGS. 2A and 2B are graphs of the amount by weight of drug (FF) in the spray-dried encapsulates determined by HPLC during different storage times;

### DETAILED DESCRIPTION OF THE PREFERRED EMBODIMENT

According to one embodiment of the present invention there is provided an encapsulated composition. The composition comprises a compound that exhibits poor water solubility, an oil, and an octenylsuccinic anhydride (OSA) modified starch. The weight ratio of starch to oil within the composition is 1:1 or less. Preferably, the weight ratio of the starch to oil is less than 1:1, about 1:1.5, 1:2, 1:2.5, 1:3, 1:3.5, or 1:4. In other embodiments, the weight ratio of starch to oil ranges from about 1:1 to about 1:4, from about 1:1.5 to about 1:3.5, from about 1:2 to about 1:3, or about 1:2.5.

In certain embodiments, the poorly water-soluble compound comprises at least 1%, 2%, 3%, 5%, 7%, 9%, or 10% by weight of the encapsulated composition. In other embodiments, the composition comprises from about 1% to about 15%, from about 3% to about 15%, from about 5% to about 12%, or from about 7% to about 10% by weight of the poorly water-soluble compound. In one or more embodiments, the poorly water-soluble compound comprises an active pharmaceutical ingredient (API), such as fenofibrate or curcumin. Although nearly any pharmaceutical compound that exhibits poor water solubility may be used. As used herein, the term “poorly water-soluble compound” refers to any compound that has a solubility in water at 250 of 10 g/L or less, and preferably less than 1 g/L, less than 100 mg/L, less than 10 mg/L, or less than 1 mg/L.

In certain embodiments, the oil comprises any oil in which the poorly water-soluble compound can be stably dispersed or dissolved, including both natural and synthetic fatty acid esters. Preferably, the oil is approved for human consumption by the U.S. FDA. In particular embodiments, the oil is a vegetable oil such as peanut oil, soybean oil, olive oil, palm kernel oil, coconut oil, or castor oil. The oil may comprise medium-chain triglycerides (MCT) having an aliphatic tail of 6 to 12 carbon atoms. The oil may also be a surfactant such as CAPRYOL 90.

The OSA modified starch is preferably that whose manufacture is described in U.S. Pat. No. 9,458,252, which is incorporated by reference herein in its entirety. Exemplary starches include those derived from corn, potato, wheat, rice, tapioca, Sago, Sorghum, waxy maize, waxy wheat, waxy potato, or high amylose corn. In certain embodiments, the starch is mixed with an organic acid anhydride reagent (e.g., octenylsuccinic anhydride) to form a reaction mixture having a neutral to alkaline pH. pH control can be achieved through the addition of a base such as sodium hydroxide, ammonium hydroxide, ammonium carbonate, or ammonium bicarbonate. The reacted starch can be dried and then heated to a temperature of at least 100° C. for a period of time to produce the modified starch.

According to another embodiment of the present invention there is provided an emulsion comprising a poorly water-soluble compound that is dispersed in an oil phase and an aqueous phase comprising OSA modified starch. The aqueous phase can be prepared by adding a quantity of OSA modified starch to water to form a starch solution comprising from about combining from about 5% to about 30% by weight, from about 7.5% to about 25% by weight, or from about 10% to about 22.5% by weight starch. The oil phase can be prepared by adding a quantity of the poorly water-soluble compound to the oil to form an oil solution comprising from about 1% to about 25% by weight, from about 2.5% to about 20% by weight, or from about 5% to about 15% by weight of the poorly water-soluble compound. In certain embodiments, the poorly water-soluble compound is provided in a crystalline form, which is then solubilized within the oil. Quantities of the starch and oil solutions are added together in amounts necessary to give the desired starch to oil ratio and then mixed to form the emulsion. In one or more embodiments, the emulsion comprises from about 50% to about 90% by weight, from about 60% to about 85% by weight, or from about 70% to about 80% by weight of the aqueous phase. In one or more embodiments, the emulsion comprises from about 5% to about 40% by weight, from about 10% to about 35% by weight, or from about 15% to about 30% by weight of the oil phase. In certain embodiments, the water phase is the continuous phase of the emulsion and the oil phase is the dispersed phase. Also, in certain embodiments, the mixing step can include a homogenization step.

The resulting emulsion exhibits good physical stability in which the oil phase droplets resist aggregation and separation for several hours. In certain embodiments, the emulsions formed comprise oil phase droplets having a mean droplet size of from about 0.05 to about 5 μm, from about 0.075 to about 2.5 μm, or from about 0.1 to about 1 μm. The mean droplet size can remain stable (e.g., within the foregoing ranges) and no separation of the emulsion into visible discrete layers for at least 1 hour, at least 2 hours, or at least 3 hours at 25° C. after emulsion formation. In certain embodiments, the emulsions exhibit a Brookfield viscosity (spindle #21), at 100 rpm shear rate and 25° C. of from about 2 to about 50 cp, from about 3.5 to about 40 cp, or from about 5 to about 35 cp.

In certain embodiments, the emulsion can be dried, preferably by a spray drying process, in which water is removed and a powder comprising the encapsulated poorly water-soluble compound. It has been observed that the poorly water-soluble compound, which originally exhibited a crystalline morphology is now amorphous in its encapsulated form. Thus, the encapsulated compound, especially when the compound is a pharmaceutical compound, exhibits a higher degree of bioavailability than the original crystalline form of the compound. In certain embodiments, the encapsulated particles exhibit a mean particle size (determined as the mean circle equivalent diameter) of from about 1 to about 100 μm, from about 1 to about 50 μm, from about 2 to about 40 μm, from about 4 to about 25 μm, or from about 7 to about 15 μm. Also, the powder remains physically stable for extended periods of time upon storage at 25° C. in that the amount of the poorly water-soluble compound present within the particles remains relatively unchanged for a period of at least 1 month, or at least 3 months, from formation of the powder.

In certain embodiments, the encapsulated powder is capable of being reconstituted into a stable emulsion upon addition of water.

### EXAMPLES

### Example 1

In this example, a starch-based drug delivery system is described. In particular, the drug delivery system is an encapsulation system to improve the solubility and bioavailability of an active pharmaceutical ingredient, such as fenofibrate (FF), in high-load drug delivery. FF was completely dissolved in medium-chain triglyceride (MCT) or Capryol 90 oil, emulsified with octenylsuccinic anhydride (OSA) modified starch and spray dried. The various formulations were prepared using different ratios of starch to oil. The emulsions were characterized by mean droplet size of 0.098-0.424 μm and viscosity of 7.8-31.8 cp. After spray drying, the drug content, density, size, and shape, and flowability of drug solid particles were determined. Spherical particles with a smooth surface were obtained from each formulation. A preferred formulation comprised 10.0% OSA starch, 20.0% Capryol 90 oil and 70% water (w/w), with a maximum solubility of FF up to 8.2% (w/w) in spray-dried encapsulates. FF in the spray dried powders was stable and remained amorphous at room temperature stored for 3 months, as confirmed by X-ray diffraction (XRD) and differential scanning calorimetry (DSC).

Octenylsuccinic anhydride (OSA) modified starch is plant based and prepared by esterification reaction of starch with OSA. Here, it is used as an emulsifier, stabilizer, and encapsulating agent due to its amphiphilic property obtained from the hydrophilicity of starch and hydrophobicity of OSA.

An objective of this study was to investigate the ability of OS starch as an emulsifier to prepare oil-in-water emulsions, and its spray-dried particles for high-loaded FF drug delivery to overcome the problem of poor aqueous solubility. OSA modified starch is prepared by a dry heating method, see U.S. Pat. No. 9,458,252. Medium-chain triglyceride (MCT) or Capryol 90 is used as oil phase. MCT is a mixture of medium chain triglycerides, mainly from caprylic (C8) and capric (C10) acids, whereas Capryol 90 is a propylene glycol monocaprylate type II. The solubility of FF in MCT and in Capryol 90 is 7.93% and 15.4%, respectively. The obtained emulsions made by micro-fluidizer and spray-dried powders were characterized for particle size distribution, viscosity, and crystallinity. The characterization of solid drug encapsulates by differential scanning calorimetry (DSC) and X-ray diffraction (XRD) would verify the crystalline state of the drug (FF) and its conversion to the amorphous state in the spray dried powders.

### Materials and Methods

Fenofibrate (purity≥99%) was obtained from Sigma-Aldrich (St. Louis, Mo., USA). Medium-chain triglyceride (MCT, Labrafac Lipophile WL 1349) and Capryol 90 were obtained from Gattefossé Co. (Paramus, N.J., USA). Octenylsuccinic acid anhydride (OSA) was obtained from Gulf Bayport Chemicals L.P. (Pasadena, Tex.). Waxy maize starch (Amioca) was provided by Ingredion Inc. (Bridgewater, N.J.). An alpha-amylase (BAN® 480 L) with an activity of 480 KNU/g was obtained from Novozymes North America, Inc. (Franklinton, N.C., USA). One KNU is defined as the amount of enzyme that dextrinizes 5.26 g of starch (Merck Amylum soluble) per hour under standard conditions (37.0° C., 0.0003MCa²⁺, and pH 5.6). All other chemicals were analytical grade.

OS starch preparation was prepared by a dry heating method. Waxy maize starch (100 g) was suspended in distilled water (150 g) with agitation. The pH of the starch slurry was adjusted by adding NH₄HCO₃. The starch suspension was filtered through filter paper (P8, Fisher Scientific, Pittsburgh, Pa.), and the starch cake was mixed with 3% OSA (wt. % based on the dry weight of starch) by a mixer (Model K45SSWH, KitchenAid, St. Joseph, Mich.) at 2nd speed for 15 min. The mixture was dried in an air-forced oven at 40° C. overnight. The starch mixture was ground by an analytical mill (A-10, Tekmar, Staufen, Germany) followed by sieving through al 10-mesh sifter. The powdered starch was thinly spread over an oven pan and heated at 180° C. for 2 h. The degree of substitution (DS) of OS starch was determined by high-performance liquid chromatography (HPLC).

OS starch (7.5, 10, 12.5, 15, or 17.5 g) in water (70 g or 60 g) was mixed and heated in a water bath at 60° C. for 6 h until the solutions were clear. See, Table 1. Fenofibrate (FF, 7 g or 15 g) was dissolved in the oil phase (100 g) at the concentration approaching the saturation concentration of FF in the oil: 7.93% in MCT and 15.4% in Capryol 90. MCT or Capryol 90 oil phase (12.5, 15.0, 17.5, 20.0, 22.5, or 30 g) was added into starch solution while mixing with a portable homogenizer (Bamix, Mettlen, Switzerland) for 3 min. The starch solution was pre-homogenized by a bench-top homogenizer (PRO 350, PRO Scientific Inc., Oxford, Conn.) at 5000 rpm for 10 min, and further homogenized by a micro-fluidizer (M-110P, Microfluidics, Newton, Mass.) for 3 passes at 30,000 psi. Particle size was measured 1, 2, 4, and 20 h after preparation of the emulsion by a laser diffraction particle size analyzer (LA-910, HORIBA, Ltd., Tokyo, Japan).

Each emulsion was dried in a spray dryer (Mini Spray Dryer B-290, BUCHI Corporation, New Castle, Del. USA) operated at an inlet temperature of 160° C. and outlet temperature of 100° C., aspirator 100%, and feed rate 15 ml/min. The collected spray dried powder was packed in a sealed glass bottle and stored at room temperature for further analysis.

TABLE 1
Formulation of fenofibrate (FF) drug emulsions using
octenylsuccinic anhydride modified starch (OS starch)

| | Ratio | Amount (g) | | | | | | Drug (FF) % |
|---|---|---|---|---|---|---|---|---|
| | (starch:oil | | OS | | Capryol | Drug (FF) | Drug (FF) % | without |
| Formulation | phase) | Water | starch | MCT oil | 90 oil | in oil | in oil | water |
| A (blank) | 1:0.7 | 70.0 | 17.5 | 12.50 | 0 | 0 | 0 | 0 |
| B | 1:0.7 | 70.0 | 17.5 | 11.68 | 0 | 0.82 | 7 | 2.7 |
| C | 1:1 | 70.0 | 15.0 | 14.02 | 0 | 0.98 | 7 | 3.3 |
| D | 1:1.4 | 70.0 | 12.5 | 16.36 | 0 | 1.14 | 7 | 3.8 |
| E | 1:2 | 70.0 | 10.0 | 18.69 | 0 | 1.31 | 7 | 4.4 |
| F | 1:3 | 70.0 | 7.5 | 21.03 | 0 | 1.47 | 7 | 4.9 |
| G | 1:3 | 60.0 | 10.0 | 28.04 | 0 | 1.96 | 7 | 4.9 |
| H (blank) | 1:0.7 | 70.0 | 17.5 | 0 | 12.50 | 0 | 0 | 0 |
| I | 1:0.7 | 70.0 | 17.5 | 0 | 10.87 | 1.63 | 15 | 5.4 |
| J | 1:1 | 70.0 | 15.0 | 0 | 13.04 | 1.96 | 15 | 6.5 |
| K | 1:1.4 | 70.0 | 12.5 | 0 | 15.22 | 2.28 | 15 | 7.6 |
| L | 1:2 | 70.0 | 10.0 | 0 | 17.39 | 2.61 | 15 | 8.7 |
| M | 1:3 | 70.0 | 7.5 | 0 | 19.57 | 2.93 | 15 | 9.7 |
| N | 1:3 | 60.0 | 10.0 | 0 | 26.09 | 3.91 | 15 | 9.7 |

Droplet size distribution of emulsions was determined using the laser scattering particle size distribution analyzer (LA-910, Horiba, Japan). All samples were performed in duplicate. The volume mean diameter was used to express the particle size and the width of particle size distribution.

The viscosity of the emulsions before and after homogenized by a micro-fluidizer was measured using a viscometer (DV-II+ Pro, Brookfield, Middleboro, Mass., USA). A shear rate 100 rpm was chosen, and tests were carried out at room temperature.

The amount of FF in the spray-dried encapsulated powder was determined by HPLC (Agilent 1100 series, Waldbroonn, Germany) equipped with a Phenomenex Kinetex Cis column (Torrance, Calif.) with 5 μm particle size. A mixture of 70% acetonitrile and 30% water was used as the mobile phase maintained at 25° C. during analysis. The flow rate was 1.0 ml/min, the injection volume was 10 μL and the detection wavelength was set at 286 nm. For FF standard curve, FF (0.1, 0.5, 1.0, 2.0 and 5.0 mg) was weighed, filled with 5 mL of ethanol, and analyzed by HPLC.

Each spray-dried encapsulate (0.1 g) was re-suspended in 0.9 mL of distilled water. α-Amylase (Ban480L, 10 μL) was added into slurry and placed into a water bath at 60° C. for 15 min. The emulsion (1 mL) was cooled to 25° C., and 9 mL of methanol was mixed with the emulsion for extraction of FF in the encapsulates. The mixture was centrifuged at 3000 g for 15 min, and the supernatant was filtered by nylon filter membranes (0.45 μm) and analyzed by HPLC. The concentration of FF was determined from the peak area and the amount of FF was calculated from FF standard curve.

Particle size distribution of spray-dried powder was determined using a microscopic image analyzer (Morphologi G3 instrument, Malvern Panalytical Inc., Westborough, Mass., USA). Circle equivalent (CE) diameter, high sensitivity (HS) circularity, convexity and elongation of each spray-dried powder were measured and recorded. CE diameter is the diameter of a circle with the same area as the projected area of the particle image. HS circularity has values in the range of 0-1, which a perfect circle has a circularity of 1 while a very “spiky” or narrow elongated object has a circularity value closer to 0. Convexity also has values in the range of 0-1. A smooth shape has a convexity of 1 while a very “spiky” or narrow elongated object has a circularity value closer to 0. Elongation is defined as [1-width/length], which also has values in the range from 0 to 1. A shape such as a circle or square has an elongation value of 0, while a rod has a high elongation (manual).

The density of the encapsulated powder samples was measured using a helium gas pycnometer (AccuPyc II 1340, Micromeritics, Norcross, Ga., USA), and was calculated from the weight and particle volume. The averages of three measurements were calculated and reported.

The angle of repose was measured by a Hosokawa powder tester (Micron powder systems, Summit, N.J.). The sample was poured into the funnel at a stable feeding by vertical vibration. After pouring the samples, the height of the cone was measured, and the angle of repose was calculated using the following relationship:

θ=arctan(2H/D) (1)

Spray-dried encapsulates (0.1 g) were dispersed with 10 mL of distilled water by handshaking, and then the solution was shaken for 10 min. The reconstituted emulsion was stored at room temperature after 3 h. Droplet size distribution of re-suspension emulsions was determined using the laser scattering particle size distribution analyzer as described above.

The physical state of the spray-dried emulsions samples was obtained with an X-ray diffractometer (APD 3520, Philips, Netherlands). The instrument was operated at 35 kV, 20 mA with Cu-Kα radiation, a theta-compensating slit, and a diffracted beam monochromator. Data were recorded between the diffraction angles (2θ) of 2° and 35°. Differential scanning calorimetry (DSC) measurement of spray dried drug powder was studied with the TA instrument Q200 V24.4 (New Castle, Del., USA) in a range of −20 to 120° C. under nitrogen flow of 70 mL/min. The spray-dried emulsions powder (3-5 mg) was accurately weighed into DSC stainless steel pan. An empty pan was used as a reference. The heating rate of 10° C./min was used.

Stability tests as mentioned above (level of FF in spray-dried powder, particle size and shape, density, flow properties, reconstitution, XRD and DSC) for all spray-dried encapsulates were performed after the storage at room temperature for 3 months.

Analysis of variance was conducted using a Statistical Analysis System (SAS, version 9.3 for Windows, SAS Institute, Cary, N.C., USA). Least significant differences for comparison of means were computed at p<0.05.

### Results and Discussion

OS starch prepared by dry heating (3% OSA, 3% NH₄HCO₃, heated at 180° C. for 2 h) was characterized and it was determined that OSA modification did not change the appearance of starch granules. The OS starch showed an A-type crystalline pattern. Compared with native waxy maize starch, the crystallinity and crystal size of OS starch decreased from 43.3% to 22.1% and 9.6 to 7.1 nm, respectively. OS starch had a lower onset temperature and lower enthalpy value measure by DSC. The DS of OS starch prepared in this study was 0.022.

Drug emulsion formulations A-G and H-N were made by OS starch (DS=0.022) with MCT oil and Capryol 90 oil, respectively, containing 7.5-17.5% (w/w) OS starch and 12.5-30.0% (w/w) of oil phase. See, Table 1. The formulations A and H were used as blank emulsions. The droplet size and the viscosity of the blank (A and H) emulsion were not significantly different with FF drug emulsions (B and I). See, Table 2. The changes in viscosity of emulsions at constant shear rate before and after homogenization by the microfludizer are presented in Table 2. Capryol 90 oil at room temperature has a lower viscosity (14.5 cp) than MCT oil (25.0 cp), and both are selected as a model oil phase for emulsion preparation in this study. Table 2 shows that the viscosity of the emulsion decreased with increasing the amount of oil and decreasing the amount of OS starch. The viscosity of emulsion with 40% solid content was higher than that with 30% at the same ratio of starch to oil (1:3). Each emulsion containing MCT (8.3-31.8 cp) was much more viscous than that containing Capryol 90 (7.8-19.0 cp) before homogenized by microfluidizer in the same ratio of starch and oil formulation due to the viscosity difference between MCT and Capryol 90 oil. After microfluidization, the viscosity of emulsions containing MCT was increased to 10.8-37.8 cp, but the viscosity of emulsions containing Capryol 90 was decreased to 5.5-13.0 cp due to their different chemical structures of oil components.

TABLE 2
Viscosity changes and particle size distribution on drug liquid emulsions after micro-fluidizer

| | Viscosity (cp) | Viscosity (cp) | | | | |
|---|---|---|---|---|---|---|
| | before micro- | after micro- | Particle size distribution (μm ± SD)³ | | | |
| Formulation¹ | fluidizer² | fluidizer² | 1 h | 2 h | 4 h | 20 h |
| A (blank) | 31.5 ± 2.5 a | 37.5 ± 0.5 a | 0.098 ± 0.002 c, A | 0.099 ± 0.000 d, A | 0.099 ± 0.003 d, A | 0.103 ± 0.004 f, A |
| B | 31.8 ± 2.5 a | 37.8 ± 2.5 a | 0.097 ± 0.003 c, A | 0.098 ± 0.003 d, A | 0.099 ± 0.001 d, A | 0.110 ± 0.001 e, B |
| C | 23.3 ± 2.5 c | 29.0 ± 2.8 b | 0.104 ± 0.003 b, A | 0.109 ± 0.004 c, A | 0.110 ± 0.003 c, A | 0.122 ± 0.004 d, B |
| D | 18.8 ± 1.8 d | 22.0 ± 2.8 c | 0.102 ± 0.002 b, A | 0.106 ± 0.003 c, A | 0.106 ± 0.004 c, A | 0.141 ± 0.003 c, B |
| E | 11.0 ± 2.1 e | 16.0 ± 0.7 d | 0.103 ± 0.004 b, A | 0.108 ± 0.003 c, A | 0.108 ± 0.004 c, A | 0.153 ± 0.002 b, B |
| F | 8.3 ± 1.8 f | 10.8 ± 1.1 e | 0.114 ± 0.003 a, A | 0.119 ± 0.008 b, A | 0.141 ± 0.002 b, B | 0.254 ± 0.004 a, C |
| G | 27.5 ± 1.4 b | 36.8 ± 1.5 a | 0.121 ± 0.005 a, A | 0.125 ± 0.009 a, A | 0.148 ± 0.003 a, B | 0.247 ± 0.006 a, C |
| H (blank) | 18.8 ± 0.5 a | 13.0 ± 0.5 b | 0.155 ± 0.000 f, A | 0.189 ± 0.007 f, B | 0.325 ± 0.000 f, C | 0.572 ± 0.003 f, D |
| I | 19.0 ± 1.0 a | 12.5 ± 0.0 c | 0.156 ± 0.006 f, A | 0.190 ± 0.008 f, B | 0.323 ± 0.002 f, C | 0.570 ± 0.003 f, D |
| J | 15.0 ± 1.0 c | 11.0 ± 0.5 d | 0.196 ± 0.005 e, A | 0.256 ± 0.006 e, B | 0.381 ± 0.000 e, C | 0.658 ± 0.004 e, D |
| K | 11.5 ± 0.5 d | 9.5 ± 0.0 e | 0.322 ± 0.009 d, A | 0.352 ± 0.006 d, B | 0.481 ± 0.002 d, C | 0.760 ± 0.002 d, D |
| L | 9.5 ± 0.5 e | 6.5 ± 0.0 f | 0.385 ± 0.006 c, A | 0.475 ± 0.009 c, B | 0.578 ± 0.007 c, C | 0.950 ± 0.008 c, D |
| M | 7.8 ± 0.3 f | 5.5 ± 0.0 g | 0.403 ± 0.000 b, A | 0.567 ± 0.001 b, B | 0.659 ± 0.002 b, C | 0.998 ± 0.005 b, D |
| N | 16.8 ± 0.3 b | 14.3 ± 0.0 a | 0.424 ± 0.005 a, A | 0.588 ± 0.000 a, B | 0.671 ± 0.000 a, C | 1.018 ± 0.018 a, D |

¹Uppercase letters (A, B, C, . . .) are the formulations of emulsions listed in Table 1.
²Viscosity was measured using Brookfield spindle #21 running at 100 rpm.
³Values are means ± standard deviations.
Different superscript lowercase letters (a, b, . . .) in each column within each formulation with same oil indicate significant differences (p < 0.05) between treatments.
Different superscript uppercase letters (A, B, . . .) in the same row indicate significant differences (p < 0.05) between storage time for each formulation.

Stable emulsions with fine particle size were obtained for all formulations. A uniform droplet size distribution (one symmetrical peak) was obtained. The average droplet diameter of fresh drug emulsions was 0.097-0.121 μm for the MCT set (formulations A-G) and 0.155-0.424 μm for the Capryol 90 set (formulations H-N). The smallest droplet size was 0.097 μm and 0.156 μm when the ratio of starch to oil was 1:0.7 in formulations B-G and I-N, respectively. The average droplet size increased with the decrease of starch concentration and the increase of oil phase concentration. At the 3:1 ratio of starch to oil, the emulsion with 40% solid content had a larger droplet size (0.121 μm for G or 0.424 μm for N) than the emulsion with 30% solid content (0.114 μm for F or 0.403 μm for M) (see, Table 2).

The average droplet size of emulsions after 1, 2, 4 and 20 h was observed to determine the stability of fresh emulsions. After emulsions were prepared, there was little change in droplet diameter for formulations A-G in 4 h, but after 20 h, droplet diameter increased to 55% for high ratio of oil amount in emulsions (formulation F and G, (0.247-0.121)/0.247=55%). However, all emulsions remained stable for 20 h and had no visible oil layer with average droplet size less than 0.247 μm for MCT set (B-G) and 1.018 μm for Capryol 90 set (I-N), respectively. Compared with formulation I-N at the storage after 20 h, droplet diameter was increased by 72% for high ratio of OS starch in emulsions (formulation I) but still small (0.57 μm). These observations showed that OS starch was effective in stabilizing oil in water emulsions.

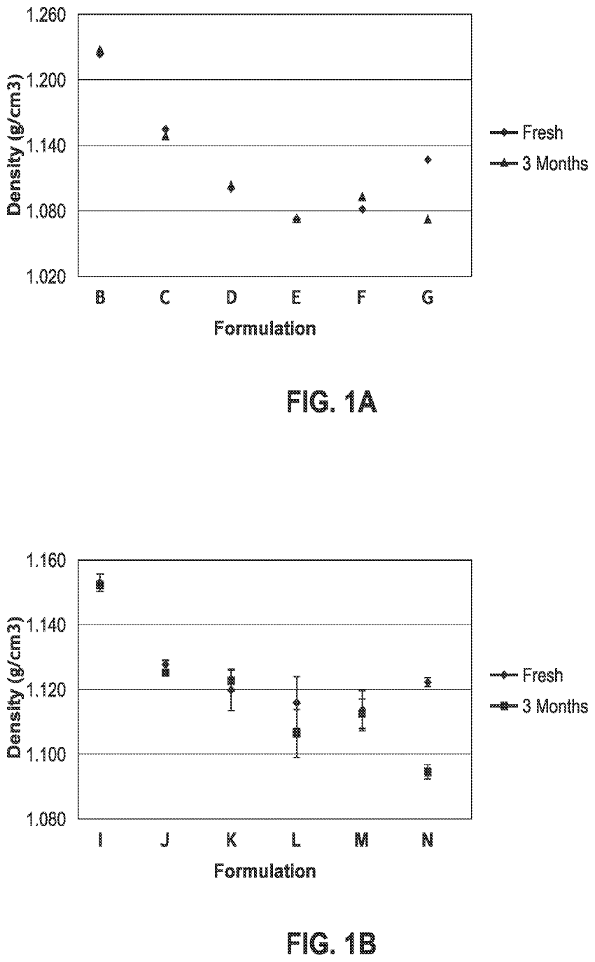

All drug encapsulates were prepared by spray drying FF emulsions using a Buchi mini spray dryer. OS starch acting as a solid carrier/wall material was sufficient to obtain the dry powder. The density of spray-dried encapsulates for MCT (formulations B-G) and Capryol 90 (formulations I-N) was 1.072 to 1.224 g/cm³, and 1.095 to 1.153 g/cm³, respectively (FIGS. 1A and 1B, Fresh), which was between the density of FF (1.25 g/cm³) and the density of OSA starch (1.06 g/cm³). After the storage of 3 months, the density of drug powder was re-measured (FIGS. 1A and 1B, 3 Months). It was found that the density remained the same among formulation B-E and I-L where the ratio of starch and oil was great than 1:2, but at the ratio of starch to oil 1:3 with 40% solid content, the density of all samples decreased to 1.072 g/cm³ for formulation G in the MCT set and 0.1095 g/cm³ for formulation N in the Capryol set, indicating that more free oil was broken out and the spray-dried particles were unstable since their densities were close to the densities of the oils (˜ 0.95 g/cm³).

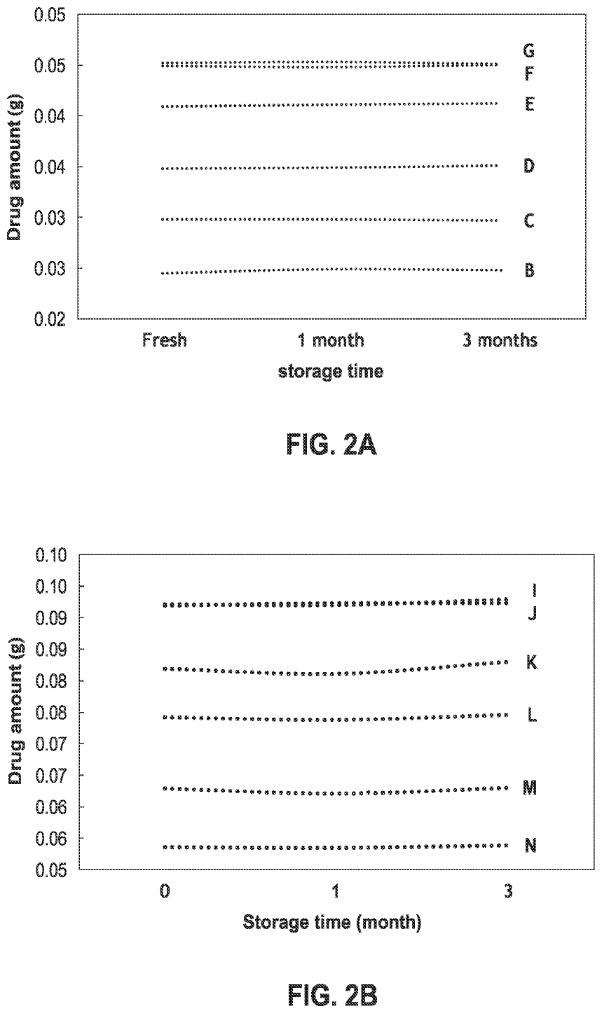

The amount of FF encapsulated in the spray-dried powders was analyzed by HPLC (see, Table 3 and FIGS. 2A and 2B). As the oil content was increased, the amount of FF was also increased, confirming that more FF was dissolved in oil phase. The FF amount in each formulation was unchanged during storage, indicating the drug in oil phase was stable in the spray-dried particles. Formulations M and N prepared from the emulsion with the ratio of starch to oil 1:3 had the highest FF content of 9.25%.

TABLE 3
Amount of fenofibrate (FF) in spray-dried
encapsulated powders as determined by HPLC.

| | Drug (g) in encapsulated powder (g)² | | |
|---|---|---|---|
| Formulation¹ | Fresh | 1 month | 3 months |
| B | 0.0245 ± 0.0005 e, A | 0.0249 ± 0.0003 e, A | 0.0248 ± 0.0004 e, A |
| C | 0.0298 ± 0.0005 d, A | 0.0298 ± 0.0003 d, A | 0.0297 ± 0.0003 d, A |
| D | 0.0348 ± 0.0002 c, A | 0.0349 ± 0.0002 c, A | 0.0351 ± 0.0005 c, A |
| E | 0.0409 ± 0.0001 b, A | 0.0411 ± 0.0002 b, A | 0.0412 ± 0.0001 b, A |
| F | 0.0449 ± 0.0003 a, A | 0.0448 ± 0.0004 a, A | 0.0450 ± 0.0002 a, A |
| G | 0.0448 ± 0.0005 a, A | 0.0455 ± 0.0002 a, AB | 0.0451 ± 0.0004 a, AB |
| I | 0.0536 ± 0.0003 e, A | 0.0535 ± 0.0002 e, A | 0.0539 ± 0.0001 e, A |
| J | 0.0629 ± 0.0006 d, A | 0.0621 ± 0.0004 d, A | 0.0630 ± 0.0002 d, A |
| K | 0.0742 ± 0.0002 c, A | 0.0738 ± 0.0004 c, A | 0.0746 ± 0.0004 c, A |
| L | 0.0819 ± 0.0002 b, A | 0.0821 ± 0.0000 b, A | 0.0820 ± 0.0007 b, AB |
| M | 0.0921 ± 0.0003 a, A | 0.0920 ± 0.0002 a, A | 0.0929 ± 0.0009 a, AB |
| N | 0.0919 ± 0.0005 a, A | 0.0923 ± 0.0001 a, A | 0.0923 ± 0.0000 a, A |

¹Uppercase letters (B, C, . . .) represent each spray-dried encapsulated powder made by the formulation of fresh emulsions listed in Table 1.
²Values are means ± standard deviations. Different superscript lowercase letters (a, b, . . .) in each column within each formulation with same oil indicate significant differences (p < 0.05) between treatments. Different superscript uppercase letters (A, B, . . .) in the same row indicate significant differences (p < 0.05) between storage time for each formulation.

The factors of particle size and shape such as circle equivalent (CE) diameter, high sensitivity (HS) circularity, convexity, and elongation were determined by the microscopic image analyzer (see, Table 4). The average particle size of encapsulates B-G ranged from 7.4 to 13.2 μm; whereas encapsulates I-N had a narrow range of average diameter from 8.6 to 10.0 μm. There was no obvious effect of the concentration of OSA starch and oil content on the particle size of all spray-dried powders. HS circularity of all samples had values in the range of 0.81-0.91, indicating the particle shape was a circle. The convexity of all samples had values in the range of 0.97-0.99, indicating a smooth shape. Elongation had values in the range from 0.15-0.25, indicating a circle shape. These three factors of particle shape were observed in all encapsulates (B-G and I-N). Therefore, the spherical particle with a smooth surface was obtained from each formulation. There were little changes of particle size and shape between fresh encapsulates and after 3 months storage.

TABLE 4
Particle size and shape, and flow properties of spray-dried encapsulates.

| | Particle size and shape² | | | | Flow behavior² | |
|---|---|---|---|---|---|---|
| | CE | HS | | | Angle of | |
| | Diameter³ | Circularity⁴ | Convexity | Elongation | repose | Flow |
| Formulation¹ | Mean (μm) | Mean | Mean | Mean | (°) | property |
| B | 8.05 c | 0.893 a | 0.990 a | 0.151 bc | 29.2 e | Excellent |
| C | 11.97 b | 0.901 a | 0.987 a | 0.176 b | 34.1 d | Good |
| D | 8.02 c | 0.895 a | 0.988 a | 0.162 b | 37.2 c | Fair |
| E | 8.42 c | 0.877 a | 0.985 a | 0.170 b | 40.2 b | Passable |
| F | 7.40 d | 0.831 b | 0.978 a | 0.233 a | 44.7 a | Passable |
| G | 15.23 a | 0.817 b | 0.968 a | 0.228 a | 45.4 a | Passable |
| I | 8.6 c | 0.847 a | 0.983 a | 0.234 a | 31.0 ab | Good |
| J | 9.11 b | 0.848 a | 0.983 a | 0.233 a | 27.5 b | Excellent |
| K | 10.01 a | 0.815 b | 0.974 a | 0.248 a | 29.2 ab | Excellent |
| L | 9.58 ab | 0.831 b | 0.977 a | 0.237 a | 32.6 ab | Good |
| M | 8.96 bc | 0.822 b | 0.974 a | 0.239 a | 30.1 ab | Good |
| N | 9.94 a | 0.820 b | 0.973 a | 0.239 a | 34.2 a | Good |
| B-3M | 6.75 d | 0.914 a | 0.993 a | 0.152 c | 30.2 e | Good |
| C-3M | 11.3 b | 0.892 ab | 0.988 a | 0.174 b | 34.9 d | Good |
| D-3M | 10.07 b | 0.87 ab | 0.986 a | 0.194 ab | 38.5 c | Fair |
| E-3M | 8.39 c | 0.853 b | 0.980 a | 0.197 ab | 44.1 b | Passable |
| F-3M | 8.55 c | 0.832 b | 0.978 a | 0.236 a | 46.7 a | Poor |
| G-3M | 14.03 a | 0.852 b | 0.975 a | 0.208 a | 47.2 a | Poor |
| I-3M | 7.59 d | 0.881 a | 0.981 a | 0.198 b | 33.1 ab | Good |
| J-3M | 7.71 d | 0.867 a | 0.987 a | 0.221 a | 31.9 b | Good |
| K-3M | 9.39 b | 0.823 b | 0.961 a | 0.245 a | 31.6 b | Good |
| L-3M | 9.87 a | 0.819 b | 0.958 a | 0.248 a | 33.3 ab | Good |
| M-3M | 9.1 c | 0.826 b | 0.961 a | 0.242 a | 34.1 a | Good |
| N-3M | 9.06 c | 0.836 b | 0.964 a | 0.229 a | 34.9 a | Good |

¹Uppercase letters (B, C, . . .) represent each spray-dried encapsulated powder made by the formulation of fresh emulsions listed in Table 1, and letters (B-3M, C-3M, . . .) represent each spray-dried powder are stored for 3 months.
²Means in the same column not sharing a common letter are significantly different at p ≤ 0.05.
³CE Diameter = Circle equivalent diameter
⁴HS Circularity = High sensitivity Circularity

The spray dried powders had angle of repose between 25°-30°, 31°-35°, 36°-40°, 41°-45°, and 46°-55°, indicating excellent, good fair, passable and poor flow property, respectively. Angle of repose is one of flow indicators that is commonly used. It can reflect the flow ability of bulk solids in an unconsolidated state and many studies showed that angle of repose has positive correlation with moisture content. A higher angle of repose could indicate poor flow of bulk material. There is no universal or standard testing method available for angle of repose, and an empirical relationship between flow properties and angle of repose was summarized by Ambrose, R. K., Jan, S., Siliveru, K., 2016, “A review on flow characterization methods for cereal grain-based powders,” Journal of the Science of Food and Agriculture 96, 359-364. The flowability of all fresh encapsulates (I-N) was better than that of encapsulates (B-G) at the same ratio of starch to oil. The results of static flow behavior (angle of repose) of encapsulates stored after 3 months were still acceptable. However, the sample F stored for 3 months (F-3M) and sample G showed poor flow behavior.

From the results of physical properties reported above, the density of G and N decreased but the drug amount had no change. To further study the stability of the encapsulated powders, drug encapsulates were reconstituted in water and evaluated for the emulsion droplet size distribution (see, Table 5). For fresh encapsulates, the mean diameter of reconstituted emulsions was obtained after shaking and there was no separation for emulsions stored for 3 h, indicating that encapsulates were stable. For the spray-dried particles reconstituted in water after 3-months storage, a cream layer was visible in sample F and G, and oil layer on water surface was visible in N, indicating that encapsulates were not stable. These results showed that the starch:oil ratio of 1:3 for MCT oil was not a good formulation to make and stabilize oil-in-water emulsions, which reached the limit of lab-made OS starch as emulsifier. However, it was found that formulation M was still stable after 3 months, indicating the starch to oil ratio of 1:3 for Capryol 90 oil with 30% solid content was an acceptable formulation.

TABLE 5
Droplet size distribution of reconstituted emulsion of fresh
encapsulated powders and those stored for 3 months.

| | | | Particle size | |
|---|---|---|---|---|
| Formu- | Ratio | Reconstitution | distribution (μm ± SD) | |
| lation¹ | (starch:oil) | condition | fresh | 3 months |
| B | 1:0.7 | encapsulated | 0.417 ± 0.03 | 0.450 ± 0.009 |
| C | 1:1 | powder:water = | 0.509 ± 0.04 | 0.498 ± 0.002 |
| D | 1:1.4 | 0.1:10 | 0.684 ± 0.01 | 0.702 ± 0.004 |
| E | 1:2 | | 1.015 ± 0.03 | 1.103 ± 0.008 |
| F | 1:3 | | 1.873 ± 0.01 | “Ring” on top |
| G | 1:3 | | 2.060 ± 0.06 | “Ring” on top |
| I | 1:0.7 | encapsulated | 0.307 ± 0.03 | 0.403 ± 0.002 |
| J | 1:1 | powder:water = | 0.450 ± 0.02 | 0.551 ± 0.002 |
| K | 1:1.4 | 0.1:10 | 0.591 ± 0.05 | 0.722 ± 0.004 |
| L | 1:2 | | 0.987 ± 0.03 | 1.167 ± 0.004 |
| M | 1:3 | | 1.450 ± 0.02 | 1.500 ± 0.005 |
| N | 1:3 | | 1.903 ± 0.02 | oil on top |

¹Uppercase letters (B, C, . . .) represent each spray-dried encapsulated powder made by the formulation of fresh emulsions listed in Table 1.

XRD and DSC were used to evaluate the crystallinity and thermal properties of spray-dried encapsulates made by OS starch and MCT or Capryol 90 oil. The crystallinity patterns of pure FF, OS starch only, mixture of OS starch and FF in the ratio of 100:10, and drug encapsulates in MCT or Capryol 90 were analyzed with X-ray diffraction. FF had sharp and high intensity peaks at diffraction angles (20) of 12.0°, 14.5°, 16.2°, 16.8°, 21.8° and 22.4°, indicating the crystalline form of the drug. In the physical mixture of OS starch and FF, sharp and high intensity peaks still appeared at the main diffraction angles (20) of 21.8° and 22.4° but the intensity was less than that of pure FF. Lab-made OS starch has no diffraction peaks indicating the amorphous structure. The absence of FF peaks was obtained in each spray-dried powder indicating the amorphous form of FF. After storage at room temperature for 3 months, XRD pattern of these spray-dried powders was re-measured. No diffraction peak appeared in any spray-dried powder samples, indicating these encapsulates were still amorphous.

DSC thermograms of pure FF, lab-made OS starch only, mixture of OS starch and FF in the ratio of 100:10, and drug encapsulates in MCT or Capryol 90 are shown in Table 6. Pure FF showed a sharp endothermic peak at a melting peak of 81.5° C. and enthalpies of 75.5 J/g which indicated that FF was crystalline. The mixture of OS starch and FF also showed a sharp endothermic peak at 81.83° C., but the peak area (ΔH=12.6 J/g) was much smaller than that of pure FF. The OS starch showed no peak indicating amorphous structure. In all spray-dried powders comprising both MCT and Capryol 90, the endothermic peak of FF disappeared, indicating the amorphous form of FF. After storage at room temperature for 3 months, DSC thermograms of these spray-dried powders was re-measured. No endothermic peak of FF at ˜ 81.5° C. appeared in any spray-dried powder samples, but there was a peak at 60-65° C. with low enthalpy ΔH<3.0 J/g for M and N, indicating these encapsulates were not amorphous for drug FF delivery. These results were used to confirm the crystalline properties of the samples with XRD. According to the emulsion preparation process, FF was stable with amorphous structure in MCT stored for three months at starch to oil ratio of 1:3 but not in Capryol 90. At starch to oil ratio of 1:2, the spray-dried powder made by OS starch and Capryol 90 was stable with amorphous structure.

TABLE 6
Thermal propertiess of fenofibrate (FF), OS starch
and FF (OSS + FF), OS starch (OSS), and spray
dried powders (M-3 M and N-3 M) stored for 3 months
as determined by differential scanning calorimetry (DSC)

| Sample | To (° C.) | Tp (° C.) | Tc (° C.) | ΔH (J/g) |
|---|---|---|---|---|
| FF | 79.7 ± 0.5 a | 81.5 ± 0.4 a | 115.0 ± 1.2 a | 75.5 ± 0.4 a |
| OSS + FF | 80.9 ± 0.6 a | 81.8 ± 0.3 a | 109.2 ± 0.8 b | 12.6 ± 0.3 b |
| OSS | 0 f | 0 f | 0 e | 0 g |
| M-3 M | 54.4 ± 0.5 c | 63.5 ± 0.4 c | 76.2 ± 1.5 d | 1.9 ± 0.1 d |
| N-3 M | 55.1 ± 0.1 b | 64.6 ± 0.1 b | 80.7 ± 0.5 b | 2.8 ± 0.1 c |

^{a}Mean ± standard deviation values are reported. Means in the same column not sharing a common letter are significantly different at p < 0.05.

In conclusion, OS starch (DS 0.022) prepared from dry heating granular starch with OSA showed excellent emulsification properties and could be used as an emulsifier and solid carrier for encapsulation of high-load drug FF delivery by spray drying. The spray dried powders containing amorphous FF was obtained. The OS starch was able to emulsify and encapsulate 200% (w/w) of oil phase (Capryol 90) and obtain spray dried powder with high level of FF (8.2%, w/w) and good flow properties. The technology reported in this study may be used as a promising approach to improve other poorly water-soluble drugs by dissolving them in an oil phase, emulsification by OS starch, and spray drying.

### Example 2

In this example, the objective was to investigate the ability of OS starch as emulsifier to prepare oil-in-water emulsions and its spray-dried particles for curcumin drug delivery to overcome the problem of poor aqueous solubility. Curcumin is classified as a biopharmaceutical classification system (BCS) class IV molecule due to its poor aqueous solubility and permeability. Curcumin has been shown to exhibit antioxidant, anti-inflammatory, antimicrobial and anticarcinogenic activities.

### Materials

Curcumin (CU) was obtained from Sigma-Aldrich (St. Louis, Mo., USA). Medium-chain triglyceride (MCT, Labrafac Lipophile WL 1349) and Capryol 90 were obtained from Gattefossé Co. (Paramus, N.J., USA).

### Methods

The same OS starch from Example 1 (15 g) was mixed in water (70 g) and heated in a water bath at 60° C. for 6 h until the solution was clear. Curcumin (CU, 1 or 5%) was dissolved in the oil phase MCT and Capryol 90. MCT or Capryol 90 oil phase (15.0 g) was added into starch solution while mixing with a portable homogenizer (Bamix, Mettlen, Switzerland) for 3 min. The starch solution was pre-homogenized by a bench-top homogenizer (PRO 350, PRO Scientific Inc., Oxford, Conn.) at 5000 rpm for 10 min. The pre-emulsion was homogenized by a micro-fluidizer (M-110P, Microfluidics, Newton, Mass.) for 3 passes at 30,000 psi. Particle size was measured 1, 2, 4, and 20 h after preparation of the emulsion by a laser diffraction particle size analyzer (LA-910, HORIBA, Ltd., Tokyo, Japan).

After measurement, the emulsion was dried in a spray dryer (Mini Spray Dryer B-290, BUCHI Corporation, New Castle, Del. USA) operated at an inlet temperature of 160° C. and outlet temperature of 100° C., aspirator 100%, and feed rate 15 ml/min. The collected spray dried powder was packed in a sealed glass bottle and stored at room temperature for further analysis.

Droplet size distribution of emulsions was determined using the laser scattering particle size distribution analyzer (LA-910, Horiba, Japan). All samples were performed in duplicate. The volume mean diameter was used to express the particle size and the width of particle size distribution.

The density of the encapsulated powder samples was measured using a helium gas pycnometer (AccuPyc II 1340, Micromeritics, Norcross, Ga., USA) and was calculated from the weight and particle volume. The averages of three measurements were calculated and reported.

The physical state of the spray-dried emulsions samples was obtained with an X-ray diffractometer (APD 3520, Philips, Netherlands). The instrument was operated at 35 kV, 20 mA with Cu-Kα radiation, a theta-compensating slit, and a diffracted beam monochromator. Data were recorded between the diffraction angles (2θ) of 2° and 35°.

Particle size distribution of the spray-dried powder was determined by using the microscopic image analyzer (Morphologi G3 instrument, Malvern Panalytical Inc., Westborough, Mass., USA). Circle equivalent (CE) diameter, high sensitivity (HS) circularity, convexity and elongation of each spray-dried powder were measured and recorded.

The angle of repose was measured by a Hosokawa powder tester (Micron powder systems, Summit, N.J.). The sample was poured into the funnel at a stable feeding by vertical vibration. After pouring the samples, the height of the cone was measured, and the angle of repose was calculated using the following relationship:

θ=arctan(2H/D) (1)

### Results

Table 7 shows the formulation of emulsions made by 15% (w/w) OS starch with 15% (w/w) MCT oil and Capryol 90 oil containing drug (curcumin, CU), respectively.

TABLE 7
Formulation of Curcumin (CU) drug emulsions using
octenylsuccinic anhydride modified starch.

| | | Amount (g) | | | | | |
|---|---|---|---|---|---|---|---|
| | Ratio | | | | | Drug | |
| | (starch:oil | | OS | MCT | Capryol | (CU) | Drug (FF) % |
| Formulation | phase) | Water | starch | oil | 90 oil | in oil | in oil |
| I | 1:1 | 70.0 | 15.0 | 14.85 | 0 | 0.15 | 1 |
| II | 1:1 | 70.0 | 15.0 | 0 | 14.28 | 0.72 | 5 |

The viscosity of emulsions I and II after being passed through the microfludizer is presented in Table 8. The viscosity of the emulsion containing MCT was 31.5 cp, much more viscous than that containing Capryol 90 (14.8 cp) due to the viscosity difference between MCT and Capryol 90 oil. A uniform droplet size distribution (one symmetrical peak) was obtained. Both of the average droplet size of fresh emulsions in MCT and Capryol 90 was small, 0.103 and 1.192 μm, respectively. In addition, the emulsions were still stable after 2 h.

TABLE 8
Droplet size distribution on drug liquid
emulsions after micro-fluidizer.

| | Viscosity (cp) | | |
|---|---|---|---|
| | after micro- | Mean droplet size (μm ± SD)^{a} | |
| Emulsion | fluidizer | 1 h | 2 h |
| I | 31.5 ± 0.5 a | 0.103 ± 0.002 a | 0.109 ± 0.002 a |
| II | 14.8 ± 0.3 a | 0.192 ± 0.002 b | 0.207 ± 0.003 b |

After spray drying, encapsulates were obtained. Spray-dried powder obtained from formulation I had a density of 1.147 g/cm³, which was higher than that obtained from II with a density of 1.126 g/cm³ (see, Table 9). The morphological properties of the particle such as circle equivalent (CE) diameter, high sensitivity (HS) circularity, convexity, and elongation were determined by the microscopic image analyzer. The average particle diameter of encapsulates I was 7.07 μm, whereas encapsulates II had a small average diameter of 6.13 μm. The morphology of particle shape was observed. HS circularity of each samples had a value at 0.892 and 0.856, respectively, indicating both of the particle shape was a circle. The convexity of two samples had a value at 0.988, indicating a smooth shape. The elongation had values at 0.172 and 0.240, respectively, indicating a shape such as a circle. Therefore, the spherical particle with a smooth surface was obtained from each formulation.

TABLE 9
Particle size and shape, and flow properties distribution of spray-dried
encapsulates (fresh: I and II; stored for 1 month: I-1M and II-1M).

| | | Particle size and shape | | | | Flow behavior | |
|---|---|---|---|---|---|---|---|
| | | CE | HS | | | Angle of | |
| | Density | Diameter¹ | Circularity² | Convexity | Elongation | repose | Flow |
| Sample | (g/cm³) | Mean (μm) | Mean | Mean | Mean | (°) | property |
| I | 1.147 a | 7.07 a | 0.892 a | 0.988 a | 0.172 a | 33.7 b | Good |
| II | 1.126 b | 6.13 b | 0.856 a | 0.988 a | 0.240 b | 28.1 d | Excellent |
| I-1M | 1.149 a | 7.05 a | 0.885 a | 0.987 a | 0.174 a | 34.5 a | Good |
| II-1M | 1.125 b | 6.15 b | 0.861 a | 0.988 a | 0.241 b | 29.5 c | Excellent |

a Mean ± standard deviation values are reported.
Means in the same column not sharing a common letter are significantly different at sp ≤ 0.05.
¹CE Diameter = Circle equivalent diameter
²HS Circularity = High sensitivity Circularity

Each spray-dried powder had an angle of repose at 33.7° and 28.1°, indicating good and excellent flow properties, respectively. There were little changes in particle density, size and shape, and flow property of the spray-dried powders after one-month storage.

The crystallinity patterns of pure CU, mixture of OS starch and FF in the ratio of 100:5, OS starch only, and drug encapsulates in MCT or Capryol 90 (I and II) between 5 and 35 of 20 were analyzed by X-ray diffraction. Pure curcumin (CU) showed several characteristic peaks at 20 angles within 30°, indicating the crystalline form of the drug. The diffractogram of the mixture of OS starch and CU exhibited a decrease in intensity at 8.9°, 12.2°, 14.5°, 17.3°, 21.1°, 24.8°, 25.7°, 27.4° and 29.0°. OS starch has no diffraction peaks indicating the amorphous structure. The absence of CU peaks was obtained in each spray-dried powder indicating the amorphous form of CU. After storage at room temperature for one month, XRD pattern of these spray-dried powders was re-measured (I-1M and II-1M). No diffraction peak appeared in any spray-dried powder samples, indicating these encapsulates were still stable for drug CU delivery in amorphous form.

1. An encapsulated composition comprising a poorly water-soluble compound, an oil, and an octenylsuccinic anhydride (OSA) modified starch, wherein the weight ratio of starch to oil within the composition is 1:1 or less, and wherein the poorly water-soluble compound comprises at least 1% by weight of the encapsulated composition.

2. The encapsulated composition of claim 1, wherein the weight ratio of starch to oil is from about 1:1.5 to about 1:3.5.

3. The encapsulated composition of claim 1, wherein the poorly water-soluble compound is an active pharmaceutical ingredient.

4. The encapsulated composition of claim 3, wherein the pharmaceutical compound comprises fenofibrate or curcumin.

5. The encapsulated composition of claim 1, wherein the oil is a vegetable oil.

6. The encapsulated composition of claim 5, wherein the vegetable oil comprises peanut oil, soybean oil, olive oil, palm kernel oil, coconut oil, or castor oil.

7. The encapsulated composition of claim 1, wherein the OSA modified starch is derived from corn, potato, wheat, rice, tapioca, sago, sorghum, waxy maize, waxy wheat, waxy potato, or high amylose corn starches.

8. The encapsulated composition of claim 1, wherein the poorly water-soluble compound is in an amorphous form.

9. The encapsulated composition of claim 1, wherein the encapsulated composition is in the form of a powder comprising particles having a mean particle size of from about 1 to about 100 μm.

10. The encapsulated composition of claim 8, wherein the amount of the poorly water-soluble compound remains substantially unchanged within the particles making up the powder for a period of at least 1 month at 25° C. following formation of the powder.

11. An emulsion comprising a poorly water-soluble compound that is dispersed in an oil phase and an aqueous phase comprising an octenylsuccinic anhydride (OSA) modified starch, wherein the weight ratio of starch to oil within the emulsion is 1:1 or less.

12. The emulsion of claim 11, wherein the aqueous phase from about 5% to about 30% by weight of the OSA modified starch.

13. The emulsion of claim 11, wherein the oil phase comprises from about 1% to about 25% by weight of the poorly water-soluble compound.

14. The emulsion of claim 11, wherein the emulsion comprises droplets of the oil phase having a mean droplet size of from about 0.05 to about 5 μm.

15. The emulsion of claim 14, wherein the mean droplet size remains stable after emulsion formation for at least 1 hour at 25° C.

16. The emulsion of claim 11, wherein the emulsion exhibits a Brookfield viscosity (spindle #21) at 100 rpm shear rate and 25° C. of from about 2 to about 50 cp.

17. The emulsion of claim 11, wherein the emulsion comprises from about 50% to about 90% by weight of the aqueous phase and from about 5% to about 40% by weight of the oil phase.

18. A method of forming an encapsulated composition comprising:
forming an oil phase comprising a poorly water-soluble compound solubilized in an oil;
forming an aqueous phase comprising an octenylsuccinic anhydride (OSA) modified starch dissolved in water;
mixing a quantity of the oil phase and a quantity of the aqueous phase to form an emulsion, wherein the weight ratio of starch to oil within the composition is 1:1 or less; and
drying the emulsion to form a powder comprising particles of the poorly water-soluble compound encapsulated in the OSA modified starch, wherein the poorly water-soluble compound comprises at least 1% by weight of the powder.

19. The method of claim 18, wherein the drying step comprises spray drying the emulsion to form the powder.

20. The method of claim 18, wherein the oil is a vegetable oil selected from the group consisting of peanut oil, soybean oil, olive oil, palm kernel oil, coconut oil, and castor oil, and wherein the poorly water-soluble compound is a pharmaceutical compound, and wherein OSA modified starch is derived from corn, potato, wheat, rice, tapioca, sago, sorghum, waxy maize, waxy wheat, waxy potato, or high amylose corn starches.

21. The method of claim 18, wherein the oil phase comprises from about 1% to about 25% by weight of the poorly water-soluble compound, and wherein the aqueous phase from about 5% to about 30% by weight of the OSA modified starch.

22. The method of claim 18, wherein the emulsion comprises droplets of the oil phase having a mean droplet size of from about 0.05 to about 5 μm, and wherein the powder comprises particles having a mean particle size of from about 1 to about 100 μm.

23. The method of claim 18, wherein the emulsion comprises from about 50% to about 90% by weight of the aqueous phase and from about 5% to about 40% by weight of the oil phase.

24. The method of claim 18, wherein the poorly water-soluble compound is in a crystalline form prior to being solubilized in the oil, and wherein the poorly water-soluble compound is in an amorphous form when encapsulated in the OSA modified starch.

25. The of claim 18, further comprising the step of dispersing the powder in water to reconstitute the emulsion.
